(19) United States
(12) Patent Application Publication
(10) Pub. No.: US 2008/0027344 A1
(43) Pub. Date: Jan. 31, 2008

(54) Modular sidestream gas sampling assembly

(75) Inventor: Randall J. Terry, Wallingford, CT (US)

Correspondence Address: MICHAEL W. HAAS;RESPIRONICS, INC., 1010 MURRY RIDGE LANE, MURRYSVILLE, PA, 15668, US

(73) Assignee: RIC Investments, LLC, Wilmington, DE (US)

(21) Appl. No.: 11/880,967

(22) Filed: Jul. 25, 2007

Related U.S. Application Data
(60) Provisional application No. 60/833,678, filed on Jul. 27, 2006.

(51) Int. Cl. A61B 5/097

(52) U.S. Cl. 600/532

(57) ABSTRACT

Apparatus and method of sampling gas from a patient using a gas sampling assembly that include a patient interface assembly having a patient interface portion and a first gas sampling tube. The patient interface portion communicates with an airway of a patient and is attached to a first end of the first gas sampling tube so that the patient interface assembly is a unitary component. A second gas sampling tube is releasably engageable with the second end of the first gas sampling tube such that substantially smooth and undisturbed fluid flow is maintained between the first gas sampling tube and the second gas sampling tube. The second gas sampling tube connects to a gas sensor and can be used with other patient interface assemblies.

### CROSS-REFERENCE TO RELATED APPLICATIONS

This application claims priority under 35 U.S.C. § 119(e) from provisional U.S. patent application No. 60/833,678, filed Jul. 27, 2006, the contents of which are incorporated herein by reference.

### BACKGROUND OF THE INVENTION

1. Field of the Invention

The present invention relates to a sidestream gas sampling gas system, and, in particular, to configuring and using the gas carrying components of such a system so that portions of the gas carrying components are disposable and other portions of the gas carrying components are reusable, thereby providing flexibility in the configuration for the sidestream gas sampling gas system while maximizing its usefulness.

2. Description of the Related Art

During medical treatment, it is often desirable to monitor and analyze a patient's exhalations to determine the gaseous composition of the exhalate. For instance, monitoring the carbon dioxide (CO₂) content of a patient's exhalations is often desirable. Typically, the carbon dioxide (or other gaseous) content of a patient's exhalation is monitored by transferring a portion, or sample, of the patient's expired gases to a suitable sensing mechanism and monitoring system.

Monitoring exhaled gases may be accomplished utilizing either mainstream or sidestream monitoring systems. In a mainstream monitoring system, the gaseous content of a patient's exhalations is measured in-situ in a patient circuit or conduit coupled to the patient's airway. In a sidestream monitoring system, on the other hand, the gas sample is transported from the patient circuit through a gas sampling line to a sensing mechanism located some distance from the patient circuit for monitoring. As a patient's expired gases are typically fully saturated with water vapor at about 35° C., a natural consequence of the gas transport is condensation of the moisture present in the warm, moist, expired gases.

FIG. 1 is a schematic diagram of a conventional sidestream gas sampling system 10. Such a system includes a gas sensor 20 and a gas sampling assembly 22 that communicates a flow of gas from the sample site to the gas sensor. Gas sampling assembly 22 is the disposable portion of the sidestream gas sampling system, meaning that a new gas sampling assembly is typically provided for each patient. It is also necessary to periodically replace gas sampling assembly 22 for the same patient as filter or dehumidifying elements in the gas sampling assembly become clogged. Gas sensor 22, however, is reused with different gas sampling assemblies.

Gas sampling assembly 22 typically includes a patient interface portion 24, a connector portion 26 that connects to gas sensor 20, and a length of hollow, flexible tubing 28 extending between the patient interface and the connector. Typically, the tubing has a length of 4-8 feet and is permanently bonded to the patient interface and the connector such that tubing 28, patient interface portion 24, and connector portion 26 are an integral unit. As a result, once gas sampling assembly 22 has reached the end of its life, it is discarded in its entirety, including the tubing, connector, and patient interface.

Accurate analysis of the gaseous composition of a patient's exhalation depends on a number of factors including the collection of a gaseous sample that is substantially free of liquid condensate, which might distort the results of the analysis. As an expired gas sample cools during transport through the gas sampling line to the sensing mechanism in a sidestream monitoring system, the water vapor contained in the sample may condense into liquid or condensate. The liquid or condensate, if permitted to reach the sensing mechanism, can have a detrimental effect on the functioning thereof and may lead to inaccurate monitoring results. Condensed liquid in the gas sampling line may also contaminate subsequent expired gas samples by being re-entrained into such subsequent samples.

In addition to the condensate, it is not uncommon to have other undesirable matter, such as blood, mucus, medications, and the like, contained in the expired gas sample. Each of these items, if present in the gas sample to be monitored, may render analytical results that do not accurately reflect the patient's medical status.

There are numerous ways in which to separate undesired matter and/or liquids from the patient's expired gas stream to protect the sensing mechanism. For instance, it is known to place a moisture trap between the patient and the sensing mechanism to separate moisture from the exhalation gas before it enters the sensing mechanism. Referring to FIG. 1, this corresponds to placing a moisture trap anywhere along gas sampling assembly 22, such as in the junction between connector portion 26 and tubing 28. The challenge, however, is to achieve the separation without affecting the characteristics of the parameters being measured, e.g., the waveform of the gas to be monitored.

By way of example, CO₂ is effectively present only in the patient's expired gases. Therefore, the CO₂ in an exhaled gas sample, which is transported through a gas sampling line to the sensing mechanism, fluctuates according to the CO₂ present at the point at which the sample is taken. Of course CO₂ levels also vary with the patient respiratory cycle. Disturbance to this fluctuation, i.e., decreases in the fidelity of the CO₂ waveform, are undesirable, because such disturbances can affect the accuracy of the CO₂ measurement and the graphical display of the CO₂ waveform. For this reason, removal of liquids and other substances from the exhaled gas sample is desirably accomplished in a way that does not substantially degrade the fidelity of the CO₂ waveform. Unfortunately, conventional moisture traps often disturb the waveform substantially.

Various techniques have been employed to filter the expired gas stream of the undesired condensate while attempting to permit the waveform to be transported undisturbed. Such techniques include absorbents, centrifugal filters, desiccants, hydrophobic membranes and hydrophilic membranes. One such technique includes making a portion of the sampling tube permeable to water vapor, for example by using dehumidification tubing, such as NAFION® brand tubing.

It is also known to provide a water trap positioned at some point along the length of the sampling tube, a water filter also positioned along the sampling tube, or any combination of the dehumidification tubing, water trap, and water filter. The effectiveness of water traps and water filters vary between manufacturers, but no water trap or water filter is immune to eventual clogging and distortion of the capnographic waveform, particularly if preventive maintenance is inadequate. Embodiments of exemplary filters suitable for sidestream gas sampling are found in U.S. patent application Ser. Nos. 11/039,749 and 11/266,864 (U.S. patent publication nos. 2005/0161042 A1 and 2006/0086254 A1), both entitled “Liquid absorbing filter assembly and system,” the contents of which are hereby incorporated by reference herein in their entirety.

Even with adequate maintenance of the breathing circuit, the “life” of existing sidestream gas sampling disposables using filters is limited by the water-holding capacity of that filter, and, as such, the cost of existing patient interfaces with integrated sidestream gas sampling limits their widespread adoption for some applications. This problem is further exacerbated by the use of a dehunified tubing such as Nafion, which is a relatively expensive component. As noted above, because the filter is integrated with the other components of the gas sampling assembly as an one-piece disposable, once the “life” of the filter is over, the entire gas sampling assembly must be discarded and replaced with a new assembly.

Given these problems with sidestream capnography systems, it is desirable to provide a cost-effective patient interface solution that (a) maintains robust performance despite the accumulation of condensate and patient secretions over the entire monitoring period, and (b) does not further degrade the fidelity of the waveform of expired gases measured.

### SUMMARY OF THE INVENTION

Accordingly, it is an object of the present invention to provide a sidestream gas sampling system that overcomes the shortcomings of conventional sidestream gas sampling systems. This object is achieved according to one embodiment of the present invention by providing a gas sampling assembly that includes patient interface assembly and a gas sensor connector assembly. The patient interface assembly includes a patient interface portion adapted to communicate with an airway of a patient and a first gas sampling tube. The first gas sampling tube has a first end, a second end opposite the first end, and a first length. The first end is coupled to the patient interface portion. The gas sensor connector assembly includes a second gas sampling tube having a first end, a second end opposite the first end, and a second length. The first end of the second gas sampling tube is releasably engageable with the second end of the first gas sampling tube such that substantially smooth and undisturbed fluid flow is maintained between the first gas sampling tube and the second gas sampling tube.

It is yet another object of the present invention to provide a method of gas monitoring a gas sample that does not suffer from the disadvantages associated with conventional gas monitoring techniques. This object is achieved by providing a gas monitoring method that includes (a) providing a first patient interface assembly that includes (1) a first patient interface portion, and (2) a first gas sampling tube having a first end coupled to the first patient interface portion; (b) providing a gas sensor connector assembly comprising: (1) a second gas sampling tube having a first end and a second end opposite the first end, and (2) a water handling component, a sample analyzing portion, or both disposed at the second end of the second gas sampling tube; (c) connecting the first patient interface assembly to the gas sensor connector assembly by connecting the first gas sampling tube to the second gas sampling tube such that a substantially smooth and undisturbed fluid flow is maintained between the first and the second gas sampling tubes; and (d) communicating a flow of gas through the first and the second gas sampling tubes to the sample analyzing portion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic diagram of a conventional sidestream gas sampling system;

FIG. 2 is a schematic diagram of a sidestream gas sampling system according to the principles of the present invention;

FIG. 3 is a schematic diagram of a sidestream gas sampling system of the present invention illustrating the flexibility in the selection of the components of the gas sampling assembly;

FIG. 4 is side view of a first embodiment of a gas sampling assembly according to the principles of the present invention;

FIG. 5 is side view of a second embodiment of a gas sampling assembly according to the principles of the present invention;

FIG. 6 is side view of a third embodiment of a gas sampling assembly according to the principles of the present invention;

FIG. 7 is a schematic view of a further embodiment of a sidestream gas sampling system according to the principles of the present invention that includes a supplemental gas delivery system;

FIG. 8 is side view of a fourth embodiment of a gas sampling assembly according to the principles of the present invention; and

FIG. 9 is side view of a fifth embodiment of a gas sampling assembly according to the principles of the present invention.

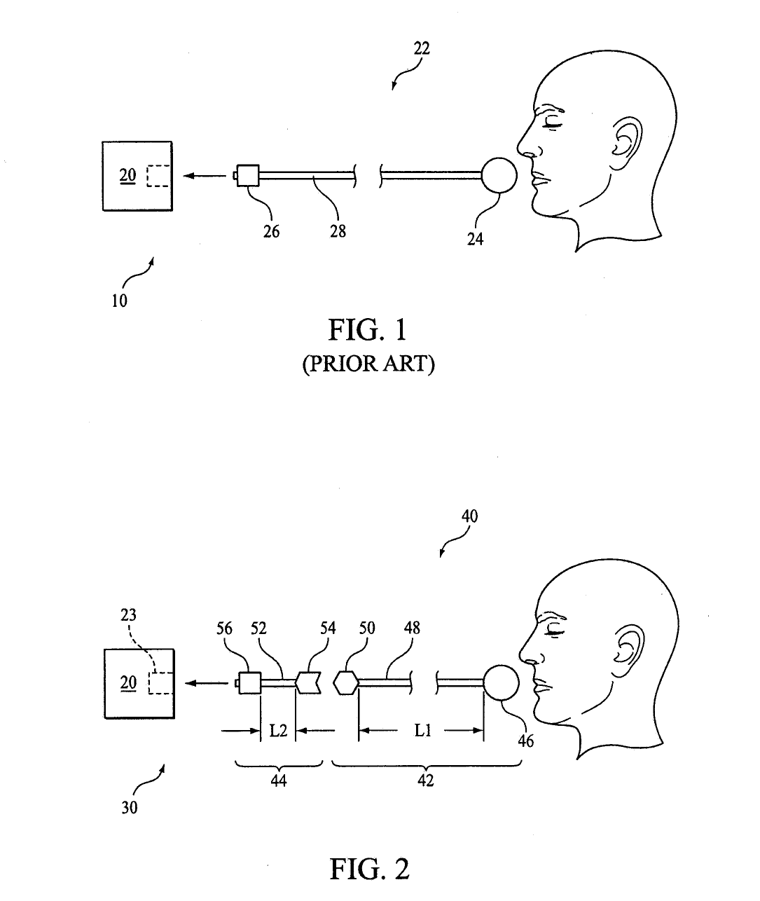

### DETAILED DESCRIPTION OF EXEMPLAR EMBODIMENTS

FIG. 2 schematically illustrates a gas carrying assembly 40 according to the principles of the present invention suitable for use in a sidestream gas sampling system 30, which includes a gas sensor 20. Gas carrying assembly 40 communicates a flow of gas, i.e., a sidestream flow of gas, from a gas sample site, such as the patient's airway or a patient circuit, to gas sensor 20 so that the constituents of the flow of gas can be measured by the gas sensor.

In the illustrated exemplary embodiment, gas carrying assembly 40 includes a patient interface assembly 42 and a gas sensor connector assembly 44, which are selectively and releasably coupled to one another to define the gas carrying assembly.

During use, patient interface assembly 42 is also coupled to the gas sample site, and gas sensor connector assembly 44 is coupled to the gas sensor.

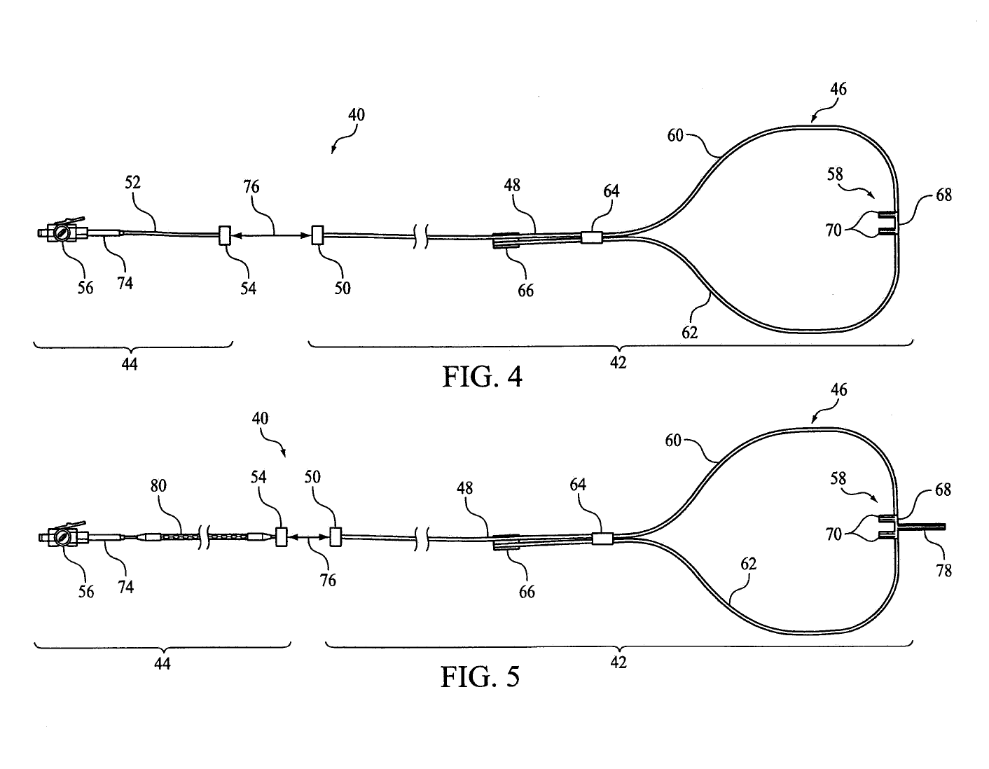

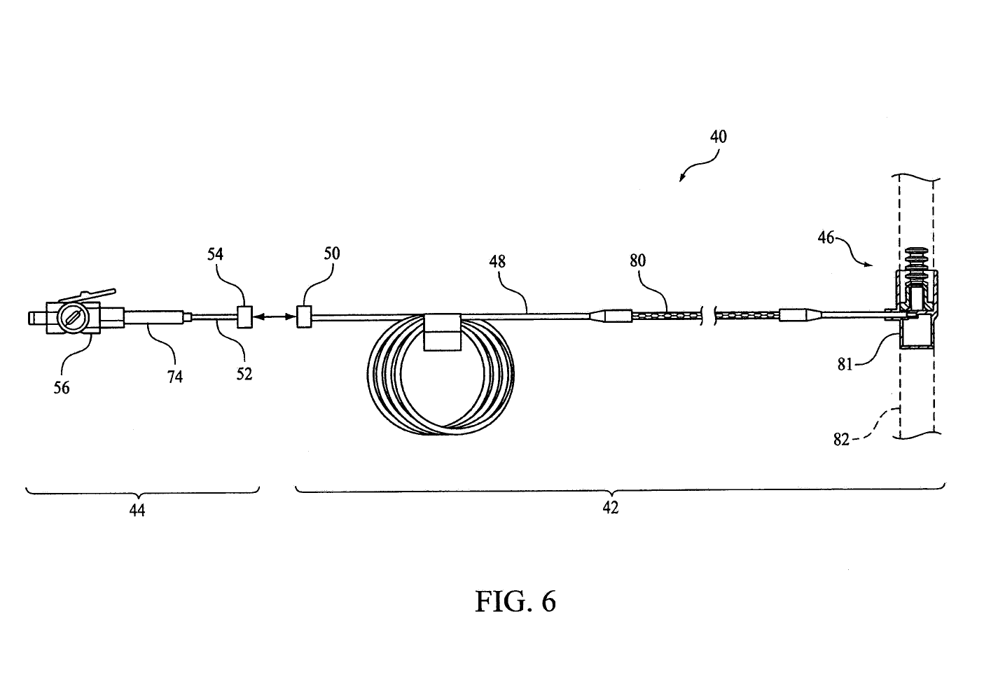

Patient interface assembly 42 includes a patient interface portion 46 that is in fluid communication with the gas sample site. In one embodiment, the gas sample site is the airway of the user. Thus, the patient interface portion is in fluid communication with the airway of the user, for example by providing a nasal cannula, oral cannula, or both. In other embodiments, the gas sample site is a patient circuit, also referred to as a breathing circuit or ventilation (vent) circuit. Thus, the patient interface portion takes the form of an airway adapter that coupled to the patient circuit. See, e.g., FIG. 6. It can thus be appreciated that the present invention contemplates a wide variety of devices can be used as the patient interface portion of the patient interface assembly, including, for example, a mask, an airway adapter (FIG. 6), a nasal cannula (FIG. 4), a nasal/oral cannula (FIG. 5), or an oral cannula.

Patient interface assembly 42 also includes a gas sampling tube 48 and a connector portion 50. Tubing 48 is a flexible tubing having a length L1, which is typically 48-144 inches (4-12 feet).

Gas sensor connector assembly 44 includes a gas sampling tube 52 having a length L2. A connector portion 54 is provided at one end of tubing 52 and a gas sensor connector portion 56 is provided at the other end. The present invention contemplates that gas sensor connector portion 56 can have any configuration suitable to connect tubing 52 to gas sensor 20. In the illustrated exemplary embodiment, gas sensor 20 includes a receptacle 23 that is sized and configured to receive at least a portion of gas sensor connector portion 56. Tubing 52 can be a flexible, rigid, semi-rigid, or any combination thereof. The present invention also contemplates eliminating tubing 52 altogether, so that gas sensor connector portion 56 and connector portion 54 are defined by a unitary element that in interposed between gas sensor 20 and patient interface assembly 42.

Connector portion 54 and connector portion 50 are configured and arranged such that they couple together and provide a substantially smooth and undisturbed fluid flow between gas sampling tube 48 and gas sampling tube 52. This smooth, undisturbed gas flow though the connection of connectors 50 and 54 is achieved, for example, by configuring the connectors such that there are no substantial changes in the inside shape or diameter of the gas flow path defined through the connectors when they are joined together.

To perform gas monitoring using gas sensor 20, the user couples patient interface assembly 42 in fluid communication with an airway of a patient, for example by attaching a nasal cannula or mask to the patient or an airway adapter in a breathing circuit to which the gas sampling tube is connected. Gas sensor connector assembly 44 is assembled with the gas sensor by attaching gas sensor connector portion 56 to the gas sensor. Patient interface assembly 42 is also assembled with gas sensor connector assembly 44 by coupling connectors 50 and 54. A pump associated with the gas sensor may be activated so that a flow of gas originating at the sample site is drawn into gas carrying assembly 40 for analysis by the gas sensor.

The present invention contemplates providing additional elements, such as filters and one or more water handling components, e.g., dehumidifiers, water traps, and the like, with patient interface assembly 42, gas sensor connector assembly 44, or both. In an exemplary embodiment, a dehumidifying element is provided in gas sensor connector assembly 44.

Configuring gas carrying assembly 40 as separate elements, i.e., patient interface assembly 42 and gas sensor connector assembly 44, achieves several benefits. The gas sensor connector assembly becomes a reusable component of the gas carrying assembly, and the patient interface assembly 42 become a disposable component. This allows the higher cost components, such as the filters and water handling elements, to be located in gas sensor connector assembly 44, which can then be used with multiple patient interface assemblies. This allows a common gas sensor connector assembly (with the associated filters and/or water handling components) to be used until its life is depleted, as opposed to having to dispose of the entire assembly, even after only short use by one patient. For example, the water-holding portion can be provided in gas sensor connector assembly 44, which can now be used on multiple patients, with each patient having their own patient interface assembly 42.

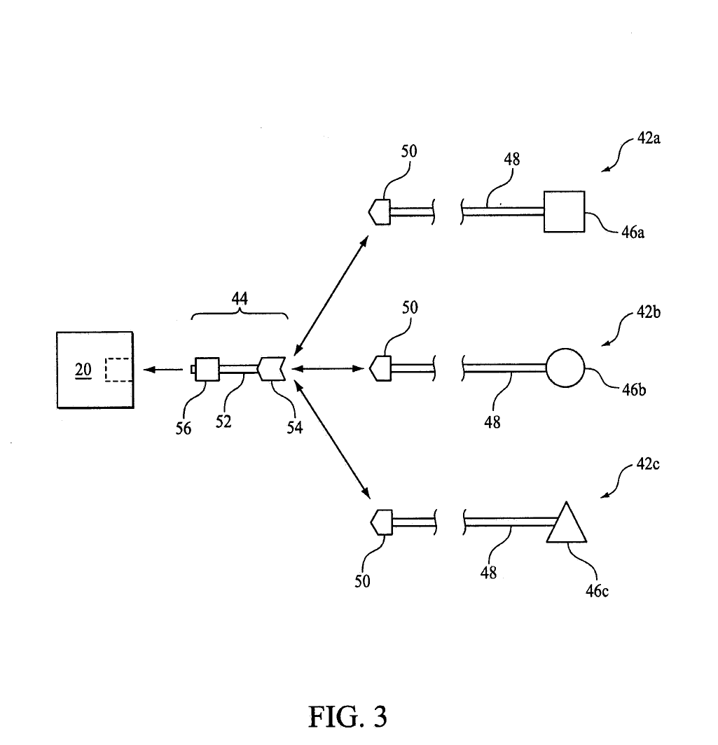

As shown in FIG. 3, this arrangement also allows different patient interface assembly 42a, 42b, and 42c, each having a different patient interface portion 46a, 46b, and 46c to be used with the same gas sensor connector assembly 44. This allows the use of one filter and/or water handling component to be used on the same patient as he or she transitions from one type of patient interface device to the next, such as from a mask, which is used in pressure support therapy and/or oxygen therapy, to a nasal cannula, which is only used in oxygen therapy and CO₂ monitoring.

In addition, as the patient is moved throughout the hospital, and is disconnected from a fixed monitoring station (gas sensor 20) the patient interface assembly can be preserved on the patient. It can then be connected to a different monitoring station, i.e., a different gas sensor using a different gas sensor connector assembly. These two gas sensor connector assemblies (44) need not have the same connection portion 56, so long as the gas sensor connector assembly has a connection portion 54 that matches connector portion 50 in the patient's patient interface assembly. Thus, the gas sensor connector assembly serves as an adapter to allow the user to move to different monitors without having to change the type of patient interface they are using.

Additionally, it is preferred to place the dehumidification tubing (given its high price) on the portion of the gas carrying assembly that can be reused the most for a particular application. For example, for long-term monitoring of the same patient (where the water-handling portion would have to be changed), it would be more cost effective to locate the dehumidification tubing on the patient interface portion. For short-term monitoring (where the patient interface portion would be frequently disposed), it would be more cost effective to locate the dehumidification tubing on the water-handling portion.

Turning now to FIGS. 4-6, various, more specific, embodiments of gas carrying assembly 40 according to the principles of the present invention will be discussed. In each of these embodiments, gas sensor connector portion 56 corresponds to the sample cell utilized in the Respironics LoFlo™ Sidestream CO₂ System. This sample cell is disclosed in U.S. patent application Ser. No. 10/384,329 (U.S. patent publication no. 2003/0191405 A1) the contents of which are hereby incorporated by reference. Gas sensor 20 includes a receptacle 23 that is sized and configured to receive at least a portion of the sample cell for securing the sample cell to the gas sensor. When sample cell 56 is assembled with the gas sensor by insertion of the sample cell at least partially into receptacle 23, one or more windows provided in the sample cell are optically aligned with the gas monitoring components of the gas monitor. Sample cell 56 includes optical windows and an optical path that allows energy to be transmitted through the flow of gas passing through the sample cell so that that the constituents of the gas passing through the sample cell can be measured.

FIG. 4 illustrates a gas carrying assembly 40 having patient interface assembly 42 that includes a loop-type of nasal cannula as patient interface portion 46. More specifically, patient interface assembly 42 includes a nasal portion 58, flexible tubing portions 60 and 62, gas sampling tube 48, a slip loop 64, an adapter 66 and connector portion 50. Nasal portion 58 includes a hollow tubular body 68 and has two nasal projections 70, each extending outwardly and adapted to fit within a corresponding nasal passage of the nose of a patient. The nasal projections provide access to the interior of the tubular body 68. Gas from the user travels along tubing 60, through tubings 48 and 52, to sample cell 56. The end of flexible tubing 62 is blocked in adapter 66. However, the present invention also contemplates connecting the end of flexible tubing 62 to tubing 48, for example using a Y-connector, so that gas from the patient flow through both tubings 60 and 62. This configuration may be more reliable as gas can flow to the sample cell even if one of tubing 60 or 62 is blocked.

Nasal portion 58 rests across the patient's nasalabial area and is held on the face of a patient by looping flexible tubing portion 60 and 62 over and behind the ears, down the jaw areas and under the chin of the patient. Although any other known means for maintaining the nasal portion on the nasalabial area and providing support for the patient interface on the face of the patient can be used. Slip loop 64 is typically of sufficient diameter to encompass both flexible tubing portions 60 and 62 and may be adjusted, i.e., moved along the length of the flexible tubing portions, so that the nasal portion remains firmly in place on the patient without the tubes being unduly taut.

Gas sensor connector assembly 44 includes sample cell 56, a water-handling component 74, second gas sampling tube 52, and connection portion 54. The water-handling component may comprise one or more of a water-holding portion and dehumidification portion. In the embodiment shown the water-handling component comprises only the water-holding portion, which is shown as filter 74. Filter 74 includes a housing typically formed of a suitable polymer, such as PVC, having a first upstream end and a second downstream end. In this embodiment, the housing is cylindrical in shape. However, it is contemplated that the housing can have any suitable shape or length.

Sample cell 56 includes a main body section, a portion of the internal volume of which forms a sample chamber in which the filtered, expired gases is collected for measurements to be taken thereof, as more fully described below. In an exemplary embodiment, the sample cell main body section is formed of polycarbonate. Sample cell 56 further includes a first side portion, a portion of an outer surface of that is coupled with second downstream end of filter 74 in a fluid-tight and gas-tight manner.

When connection portion 54 connected to connector portion 50, as shown by arrow 76, a tubing connection is formed. Tubing connection is a fluid-tight and gas-tight arrangement, as known to those skilled in the art. The mode of connecting connector portions 50 and 54 includes female/male connection and well as any known releasably fastenable mode of connection, including but not limited to a pneumatic coupling with a latch that provides for single-handed operation and an audible “click”. Connector portions 50 and 54 may also have any shape, size, or configuration so long as the function of releasably fastening the ends of tubes 48 and 52 is achieved while also providing a substantially smooth and undisturbed fluid flow between gas sampling tubes 48 and 52.

By way of example, and not by way of limitation, flexible tubing portions 60 and 62 can have an overall length of from about 15 to about 30 inches, preferably about 24 inches, although any length is suitable. The total length of the first gas sampling tube 48 and second gas sampling tube 52, i.e., L1+L2, can be from about 75 inches to about 100 inches, more preferably about 96 inches, although the length can vary depending upon the application.

FIG. 5 illustrates an embodiment of a gas carrying assembly 40 that is substantially similar to that shown in FIG. 4. However, in the embodiment of FIG. 5, patient interface portion 46 is a nasal/oral interface. That is, nasal portion 58 further includes an oral sampling portion 78 that is adapted to receive a flow of gas existing the patient's mouth. Also, gas sensor connector assembly 44 includes a dehumidification portion 80. In the illustrated exemplary embodiment, dehumification portion 80 is a length of dehumidification tubing, which is typically 2-3 inches. However, other lengths for the dehumidification tubing are contemplated by the present invention. One example of such dehumidification tubing is NAFION®, which is a DuPont co-polymer that is highly selective in the removal of water vapor. The water moves through the membrane wall and evaporates into the surrounding air or gas in a process called perevaporation.

This process is driven by the humidity gradient between the inside and the outside of the tubing.

The embodiment of gas carrying assembly 40 shown in FIG. 6 is also similar to that of FIGS. 4 and 5 except that patient interface portion 46 is an airway adapter 81, which is placed in a patient circuit 82. In the illustrated embodiment, airway adapter 81 is a low-deadspace airway adapter for use with neonatal/infant patients. However, any airway adapter, which interfaces with a patient breathing circuit from which gas may be sampled, is contemplated for use with a separable patient interface. Examples of suitable airway adapters are disclosed in U.S. Pat. Nos. 7,059,322 and 6,935,338, the contents of which are incorporated herein by reference.

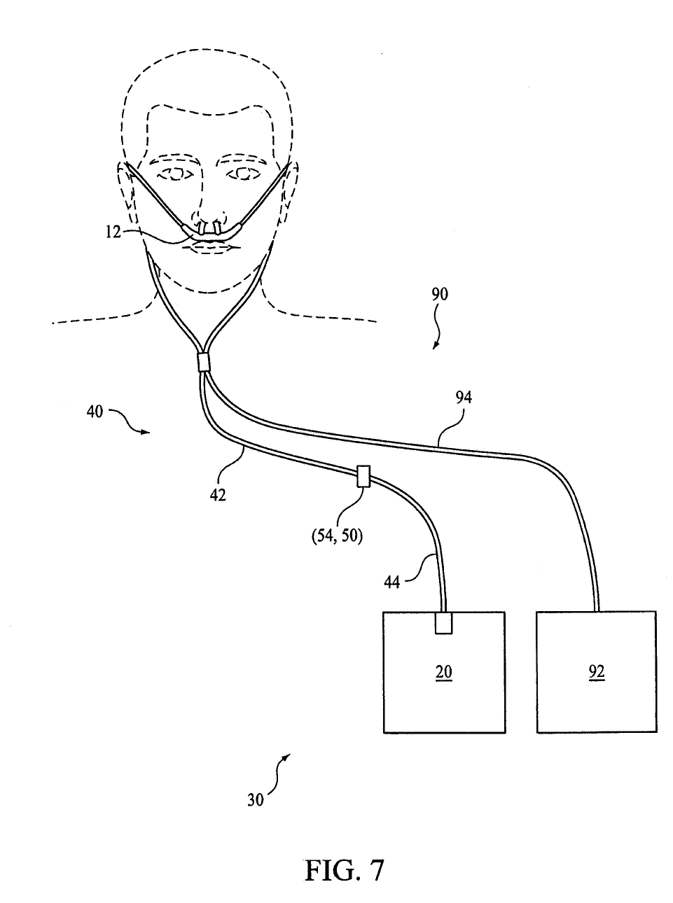

In addition to monitoring the gas exhaled by a user, the present invention contemplates providing a supplemental gas, such as oxygen, helium, nitrogen, or any combination thereof (e.g., heliox) to the user. FIGS. 7-9 illustrate a sidestream gas sampling system 30 and components thereof according to the principles of the present invention that includes a supplemental gas delivery system 90 to accomplish this function. As shown in FIG. 7, supplemental gas delivery system 90 includes a gas source 92 and a supplemental gas delivery tubing 94 that carries the supplemental gas. In the illustrated embodiment, the flow of supplemental gas is communicated to patient interface portion 46 of gas carrying assembly 40.

The gas source can be any type of gas supply. For example, in an oxygen delivery system, the oxygen source may include, but is not limited to: (a) compressed oxygen stored as a gas in a tank, (b) liquid oxygen stored in a large stationary tank that stays in the home or generated in the home, and (c) oxygen extracted from room air using any conventional gas separation technique, such as pressure swing absorption typically provided by an oxygen concentrator. A gas conserving device, such as a demand inspiratory flow system or pulsed-dose gas delivery system can also be used to control the flow of gas delivered to the user.

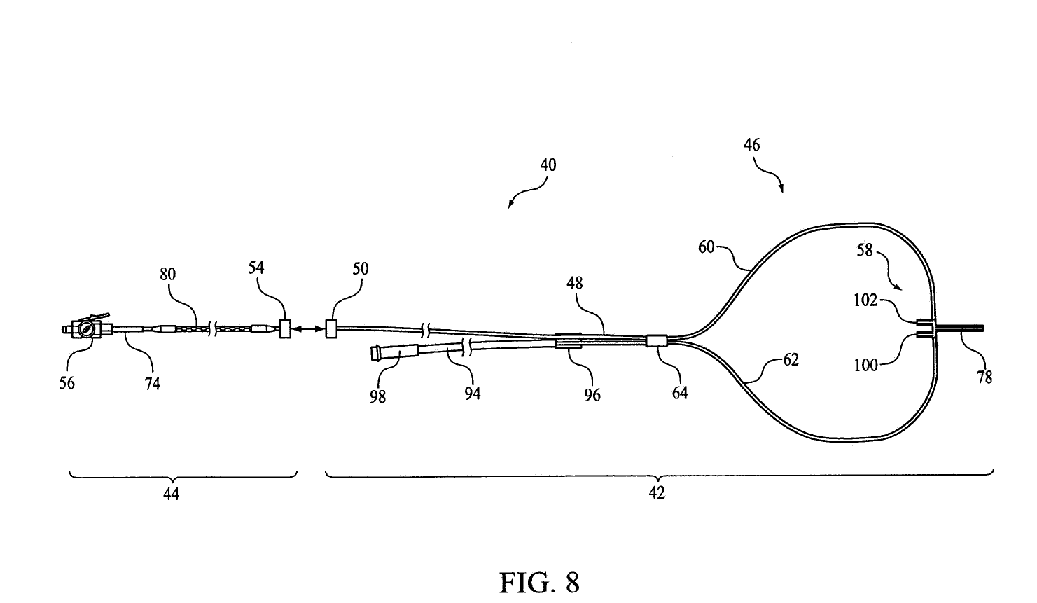

FIG. 8 illustrates a gas carrying assembly 40 that includes the gas monitoring and gas delivery capabilities. Gas carrying assembly 40 is generally similar to that of previous embodiments except for the addition of supplemental gas delivery tubing 94. In this embodiment, one end of supplemental gas delivery tubing is coupled to flexible tubing 62 via adapter 96. The other end of supplemental gas delivery tubing includes a connection portion 98 that allows the supplemental gas delivery tubing to be selectively coupled to an outlet of the oxygen source. The present invention also contemplates selectively connecting the end of supplemental gas delivery tubing to adapter 96 and providing a cap for the gas delivery portion of the adapter so that this gas carrying assembly has the flexibility of being used as a monitoring-only assembly or as both a monitoring and gas delivery assembly. In addition, connector portions similar to connector portions 50, 54 can be provided anywhere along the length of supplemental gas delivery tubing 94 so that this tubing can be separated into multiple sections.

Nasal projection 100 is coupled to flexible tubing 62 to communicate the supplemental gas to one of the patient's nares. The other nasal projection 102 receives the gas from the user. Nasal projections are physically isolated from one another so that that gas monitoring and gas delivering can be provided by one pair of nasal projections. For example, a wall, diaphragm, or other occlusion can be provided in tubing 60, 62 between (a) nasal projection 100 and (b) nasal projection 102 and oral sampling portion 102 to act as a barrier separating the gas sampling portions 102, 78 of the nasal interface portion from the gas delivery portion 100. In this embodiment, only nasal projection 100 serves as the gas delivery portion. The other nasal projection 102 and oral sampling portion 78 serves as the nasal gas sampling portion, both of which are physically isolated from gas delivery nasal projection 100.

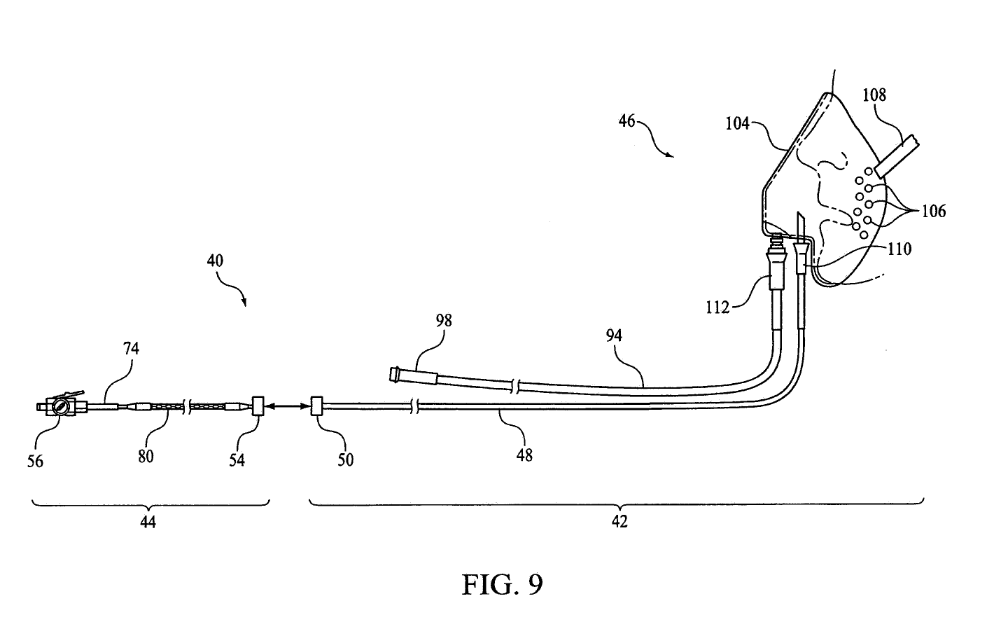

FIG. 9 illustrates another embodiment for a gas carrying assembly 40 in which patient interface portion 46 includes a mask 104, which is placed on the face of a patient. Mask 104 typically covers the nose and mouth of the user and includes exhaust ports 106 defined in the mask shell to allow gas to escape to the ambient atmosphere. It is to be understood that any conventional mask, including masks used to provided a pressure support therapy, can be used in this embodiment. A optional headgear strap 108 is provided to maintain the mask on the user.

Gas sampling tube 48 is connected to mask 104 via a gas sampling connecting portion 110. This allows the gas sampling tube to be selectively connected to the mask. However, the present invention also contemplates integrating the end of gas sampling tube 48 into the mask. Supplemental gas delivery tubing 94 is connected to the mask via a gas delivery connection portion 112. However, the present invention also contemplates integrating the end of supplemental gas delivery tubing 94 into the mask.

In an exemplary embodiment, mask 104 is a low deadspace oxygen delivery/gas sampling mask that is placed over both the nasal and oral portions of the face. However, any mask, which may be adapted for gas sampling, is contemplated for use with a separable patient interface. Nasal masks, face masks, full face masks and other means of interfacing to the patient are contemplated. These masks may be used for monitoring as well as therapies such as oxygen, aerosol and non-invasive positive ventilation therapies.

The present invention contemplates that connector portions 50 and 54 include electrical or optical connections so that electrical or optical contact can be made between patient interface assembly 42 and gas sensor connector assembly 44. This allows one or more electrical components, such as temperature sensors, humidity sensor, microphones, oximetry sensor, plethysmography sensors, motion sensor, etc., to be provided in patient interface assembly 42 in hardwired communication with the gas sensor via gas sensor connector assembly 44.

In an exemplary embodiment, the gas carrying assembly of the present invention is provided in the form of a kit that is contained in a common packaging. The kit includes patient interface assembly 42 and gas sensor connector assembly 44. A dehumidification tubing can also be provided that selectively coupled to either the patient interface assembly and gas sensor connector assembly. In a further embodiment, the kit includes one gas sensor connector assembly 44 and multiple patient interface assemblies, each with a different patient interface portion, so that the user can select which patient interface assembly to use. Other kits can include one or more patient interface assemblies, so that once the gas sensor connector assembly is put in place, other patient interface assemblies having the same or different patient interface portion can be made available to the user.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of gas monitoring comprising the steps of:
(a) providing a first patient interface assembly comprising: (1) a first patient interface portion, and (2) a first gas sampling tube having a first end, a second end opposite the first end, and a first length, wherein the first end is coupled to the first patient interface portion;
(b) providing a gas sensor connector assembly comprising: (1) a second gas sampling tube having a first end, a second end opposite the first end, and a second length, and (2) a water handling component, a sample analyzing portion, or both the water handling component and the sample analyzing portion disposed at the second end of the second gas sampling tube;
(c) connecting the first patient interface assembly to the gas sensor connector assembly by connecting the first gas sampling tube to the second gas sampling tube such that a substantially smooth and undisturbed fluid flow is maintained between the first and the second gas sampling tubes; and
(d) communicating a flow of gas through the first and the second gas sampling tubes to the water handling component, the sample analyzing portion, or both the water handling component and the sample analyzing portion.

2. The method of claim 1, further comprising:
(e) disconnecting the first patient interface assembly from the gas sensor connector assembly by disconnecting the first gas sampling tube from the second gas sampling tube;
(f) providing a second patient interface assembly comprising: (1) a second patient interface portion, and (2) a third gas sampling tube having a first end, a second end opposite the first end, and a third length, wherein the first end is coupled to the second patient interface portion;
(g) connecting the second disposable assembly to the gas sensor connector assembly by connecting the third gas sampling tube to the second gas sampling tube such that a substantially smooth and undisturbed fluid flow is maintained between the third and the second gas sampling tubes;
(hi) communicating a flow of gas through the third and the second gas sampling tubes to the sample analyzing portion; and
(i) measuring a property of a flow of gas in the sample analyzing portion.

3. The method of claim 1, further comprising:
(e) removing moisture from the first gas sampling tube, the second gas sampling tube or both the first and the second gas sampling tubes;
(f) filtering the gas flow in the first gas sampling tube, the gas flow in the second gas sampling tube, or both gas flows in the first and the second gas sampling tubes; or
(g) both steps (e) and (f).

4. The method of claim 1, wherein the patient interface portion comprises a mask, an airway adapter, a nasal cannula, a nasal/oral cannula, or an oral cannula.

5. The method of claim 1, further comprising delivering a supplemental gas to patient interface portion.

6. The method of claim 1, further comprising:
(e) disconnecting the first patient interface portion from the first gas sampling tube; and
(f) connecting a second patient interface portion to the first gas sampling tube.

7. The method of claim 1, wherein the first patient interface assembly includes a first connector coupled to the second end of the first gas sampling tube, wherein the gas sensor connector assembly includes a second connector coupled to the first end of the second gas sampling tube, and wherein connecting the first patient interface assembly to the gas sensor connector assembly includes engaging the first connector with the second connector.

8. A gas sampling assembly comprising:
(a) a patient interface assembly comprising:
(1) a patient interface portion adapted to communicate with a gas sample site, and
(2) a first gas sampling tube having a first end, a second end opposite the first end, and a first length, wherein the first end is coupled to the patient interface portion; and
(b) a gas sensor connector assembly comprising:
(1) a second gas sampling tube having a first end, a second end opposite the first end, and a second length, wherein the first end of the second gas sampling tube is releasably engageable with the second end of the first gas sampling tube such that substantially smooth and undisturbed fluid flow is maintained between the first gas sampling tube and the second gas sampling tube, and
(2) a gas sensor connector portion disposed as the second end of the second gas sampling tube, wherein the connector is adapted to couple the gas sensor connector assembly to a gas sensor.

9. The assembly of claim 8, wherein the patient interface portion comprises a mask, an airway adapter, a nasal cannula, a nasal/oral cannula, or an oral cannula.

10. The assembly of claim 8, further comprises a water-handling component operatively coupled to the first gas sampling tube, the second gas sampling tube, or both

11. The assembly of claim 10, wherein the water-handling component comprises a water-holding portion, a dehumidification portion, or a combination thereof.

12. The assembly of claim 11, wherein the water-holding portion comprises a filter, a trap, or a combination thereof.

13. The assembly of claim 12, wherein the filter comprises one or more of a hydrophilic component or a hydrophobic component.

14. The assembly of claim 11, wherein the dehumidification portion comprises tubing permeable to water vapor.

15. The assembly of claim 8, further comprising a gas delivery tubing having a first end in fluid communication with the patient interface portion and a second end adapted to be interfaced to a gas delivery system.

16. The assembly of claim 8, further comprising a first connector coupled to the second end of the first gas sampling tube and a second connector coupled to the first end of the second gas sampling tube, wherein the first connector and the second connector are configured to engage one another.

17. The assembly of claim 8, further comprising a sample analyzing portion disposed at the second end of the second gas sampling tube.

18. The assembly of claim 17, wherein the sample analyzing portion comprises a sample cell, and further comprising:
a gas sensor operatively coupled to the sample cell so as to outputs a signal indicative of a property of a gas in the sample cell; and
a processing element adapted to receive the signal and to determine a respiratory gas variable based on the signal.

19. The assembly of claim 8, wherein the first length is at least five times greater than the second length.

20. The assembly of claim 8, wherein the first length is 48-144 inches and the second length is 1-7 inches.
